(19) United States
(12) Patent Application Publication
(10) Pub. No.: US 2011/0015620 A1
(43) Pub. Date: Jan. 20, 2011

(54) TENSIONING SYSTEM

(76) Inventor: Panagiotis Oikonomidis, Thessaloniki (GR)

Correspondence Address: HOFFMANN & BARON, LLP, 6900 JERICHO TURNPIKE, SYOSSET, NY, 11791, US

(21) Appl. No.: 12/811,557

(22) PCT Filed: Jan. 13, 2009
(86) PCT No.: PCT/GR2009/000002

§ 371 Date: Oct. 5, 2010

(30) Foreign Application Priority Data
Jan. 16, 2008 (GR) 20080100022

(51) Int. Cl. A61B 18/20; A61B 18/18

(52) U.S. Cl. 606/9; 606/33

(57) ABSTRACT

An apparatus and a method for stretching a biaxially or radially deformable, resilient, flat or curved surface, comprising a radiation supplying means, such as intense pulsed light (IPL) or laser radiation, having an end portion through which said radiation is supplied to a radiation receiving part of said surface, and a handpiece comprising at its tip at least one anchoring means to be applied to said surface, said anchoring means being positioned laterally to said end portion and being operable to move in the direction of at least one of the axes of said biaxially or radially deformable, flat or curved surface and away from said radiation receiving part of said surface. In operation, said movement of the anchoring means away from said radiation receiving part of said surface can be achieved by pressing said anchoring means against said surface. By tensioning the surface at the periphery of the radiation receiving part, the end of the optical element at the end portion of the radiation supplying means does not contact the radiation receiving surface and leaves enough space for the supply of cooling air.

### BACKGROUND OF THE INVENTION

The present invention is related to the field of skin treatment based on irradiation with light or another electromagnetic radiation, for instance with intense pulsed light (IPL) or with laser radiation. In particular, the invention is related to an apparatus and a method for stretching (tensioning) a biaxially or radially deformable flat or curved surface, such as human skin, comprising a hand piece adapted to the end portion of an electromagnetic radiation supplying means, said hand piece being applied to the surface of the skin to be treated.

The use of electromagnetic radiation for the treatment of skin disorders under the skin surface is known as a non-invasive skin treatment, wherein light is selectively absorbed by unwanted hair shafts, blood vessels, pigmented lesions or pigmented stains present in the skin either by nature (high melanin concentration), or caused by the exposure of the skin to ultraviolet radiation by sun tanning or, for instance, generated artificially by the creation of tattoos.

The treatment is accordingly directed to the destruction of unwanted hairs, coagulation of blood vessels of e.g. spider veins in the legs, treatment of pigmented skin either in the form of an erasure of dark sun stains or a tattoo removal. The treatment with electromagnetic radiation is based on the selective energy absorption of the hair, which is darker than the surrounding skin, or the selective energy absorption of the pigments present in dark sun stains or tattoos.

The main commercial application of the aforementioned exposure of skin to electromagnetic radiation is cosmetic skin treatment and in particular hair removal. Hair removal can be practiced either by physicians or, in recent years, by cosmetic studios. It is noted, though, that the application of laser devices is constrained to use only by physicians whereas the use of intense pulsed light (IPL) devices is also allowed to the personnel of cosmetic studios.

One of the problems encountered during the application of electromagnetic radiation is the generation of heat at the spot targeted by the radiation, in particular when the radiation is a laser beam at the infrared wavelength. As a consequence, the treatment can not only be very painful but in addition thereto the heated skin suffers damage of the tissue. The problem has therefore been considered to be solved by cooling of the skin. Various methods for cooling have been taken into consideration.

For instance, spraying of cooling gases onto the skin during the irradiation has been widely used, e.g. hydrofluorocarbons (HFC) like Freon 134a (HFC 134a). Hydrofluorocarbons (also called fluorinated hydrocarbons) are however harmful to the skin because they allow very deep cooling and often cause frostbite at the region of the skin where they are applied. Another drawback of the hydrofluorocarbons is that they cause health damage when inhaled. Finally, since it was found that they deplete the ozone layer of the atmosphere resulting at global warming, therefore being harmful for the environment, they were classified as fluorinated greenhouse gases as covered by the Kyoto Protocol and the prevention and minimization of their emissions were regulated by the European Regulation EC 842/2006 of 17 May 2006, published in the Official Journal of the European Union. There is therefore a need to develop a cooling method avoiding the use of harmful hydrofluorocarbons.

Cooling by contacting the skin has also been taken into consideration. This cooling method involves contacting the skin with a metallic device prior to its exposure to electromagnetic radiation. The disadvantage of this method is that the devices used for cooling are opaque and do not allow simultaneous cooling and exposing the skin to the radiation. The method is therefore discontinuous, i.e. the irradiation must be interrupted for several seconds prior to the metallic device contacting the next region of the skin to be treated. Thus it is particularly time consuming and inefficient. Furthermore, the metallic surface often adheres to the skin, since skin moisture freezes and sticks to it, also causing frostbites.

A further approach was made to direct the IPL or laser beam through a cooled transparent medium in the form of two planar glass plates, one facing towards the light source and one contacting the skin to be treated. A cooling liquid was allowed to flow between these glass plates. The beam was directed to be perpendicular to the glass surfaces and was targeted to the skin surface after passing through the first (upper) glass plate, the cooling liquid and the second (lower) glass plate contacting the skin.

The disadvantages of this cooling device are the formation of condensation drops on the surface of the first (upper) glass plate due to humidity in the air and the deposition of skin residues, skin fat and particles on the bottom of the second (lower) glass plate contacting the skin. In view of the deposition of these unwanted substances on both surfaces of the cooling device, the glass plates become opaque and affect its transparency and its transmission properties, resulting in a significant absorption of the light emitted by the IPL or laser beam source. A higher consumption of energy and a frequent wiping and cleaning of the glass surfaces are necessary, disrupting the continuity of the irradiation process.

In a further approach, apparatuses have been developed, with which pressure is provided to the skin by contacting it with the end of the optical element through which the skin is irradiated. The pressure is perpendicular to the skin and is applied by direct contact to it. In this case cooling is effected by cooling the end of the optical element which contacts the skin surface.

Several approaches were made, directed to cooling by cold air supply; the cooling capacity was however not proven sufficient for an effective and painless application of intense pulsed light (IPL) or laser radiation to the skin.

### SUMMARY OF THE INVENTION

The object of the present invention is the provision of an effective and environmentally viable method and an apparatus for stretching a biaxially or radially deformable, resilient, flat or curved surface such as human skin, for it to be simultaneously exposed to a radiation source such as intense pulsed light (IPL) or laser radiation.

The present invention is based on the finding that skin stretching or tensioning reduces the concentration of pigments on the surface of the skin and the blood content in the vessels beneath the surface of the skin and that by reducing them the skin gets lighter coloured and absorbs less heat, thus significantly reducing the cooling needs. The simultaneous cooling can be performed by supplying a cooling means, e.g. air or a cooling gaseous or spray composition.

A first embodiment of the invention provides an apparatus for stretching a biaxially or radially deformable, resilient, flat or curved surface according to claim 1, said apparatus comprising:
an electromagnetic radiation supplying means having an end portion through which said radiation is supplied to a radiation receiving part of said surface, and
a handpiece comprising at its tip at least one anchoring means to be applied to said surface

said anchoring means being positioned laterally to said end portion

and being operable to move in the direction of at least one of the axes of said biaxially or radially deformable flat or curved surface and away from said radiation receiving part of said surface.

In a second embodiment the present invention provides a method for stretching a biaxially or radially deformable, resilient, flat or curved surface according to claim 18, said method comprising the steps of:
providing an electromagnetic radiation supplying means having an end portion through which said radiation is supplied to a radiation receiving part of said surface,
providing a handpiece at said end portion of the electromagnetic radiation supplying means, said handpiece comprising at its tip at least one anchoring means,
applying said at least one anchoring means to said surface,

said at least one anchoring means being positioned laterally to said end portion,

and further comprising the step of operating said at least one anchoring means to move in the direction of at least one of the axes of said biaxially or radially deformable flat or curved surface and away from said radiation receiving part of said surface.

In operation, said movement of the anchoring means away from said radiation receiving part of said surface can be achieved by pressing said anchoring means against said surface.

In a third embodiment the invention provides a use of an apparatus according to the first embodiment for treatment of the skin.

Preferred embodiments are defined in the dependent claims of the present specification.

One of the main advantages of the present invention is that by tensioning the surface by anchoring means positioned at the periphery of the radiation receiving part and by moving said anchoring means in the direction of at least one of the axes of said surface, i.e. on the plane of the targeted surface itself, the end of the optical element at the end portion of the electromagnetic radiation supplying means does not contact the radiation receiving surface and leaves enough space for the supply of cooling air.

### BRIEF DESCRIPTION OF THE DRAWINGS

The invention is illustrated by example in the accompanying drawings.

FIG. 1a is a lateral view of the apparatus according to one embodiment of the invention, wherein the anchoring means of the handpiece comprises a vacuum applying means comprising a first and a second member being positioned such as to face each other.

FIG. 1b is a bottom view of the embodiment shown in FIG. 1a.

FIG. 2a is a lateral view of the apparatus according to another embodiment of the invention, wherein the anchoring means of the handpiece comprises abrasive microprotusions, e.g. abrasive paper, as a first and a second member being positioned such as to face each other.

FIG. 2b is a bottom view of the embodiment shown in FIG. 2a.

FIG. 3 is a bottom view of still another embodiment of the invention, wherein the plural anchoring means of the handpiece are arranged as two couples, each comprising a first and a second member being positioned such as to face each other.

FIG. 4 is a bottom view of another embodiment of the invention, wherein the plural anchoring means comprises a first and a second member in curvilinear form, being positioned such as to face each other.

FIG. 5 is a bottom view of another embodiment of the invention, wherein the plural anchoring means are arranged as two couples, each comprising a first and a second member in circular form, being positioned such as to face each other.

FIG. 6 is a three dimensional view of the apparatus according to the embodiment shown in FIGS. 1a and 1b.

FIG. 7 is a three dimensional view of an apparatus according to an embodiment of the invention as it is positioned by finger pressure on the skin target.

FIG. 8a is a depiction of the apparatus immediately after its application to the skin prior to stretching and FIG. 8b after the skin target is stretched.

FIG. 9 is a lateral view of another embodiment, wherein the anchoring means is rotatable around an axis.

FIGS. 10 and 11 show the application of the latter embodiment to the skin target, prior and after the skin is stretched.

FIG. 12 is a lateral view of an embodiment, wherein a major part of the apparatus is shown, including the electromagnetic radiation supplying means and the cooling device comprising a nozzle for supplying cooling air.

FIG. 13 (Diag.1) is a graph of deformation of the stretched skin surface versus the applied tension.

FIG. 14 (Diag.2) is a diagram showing the synchronization of the intermittently applied vacuum with the cycles of light pulses emitted by the electromagnetic radiation supplying means (the light source).

### DETAILED DESCRIPTION OF THE INVENTION

According to a preferred embodiment of the invention the anchoring means is operable to generate a tension in a direction of said surface in the range of about 0.01 to about 10 MN/m² (meganewton per square meter) and preferably in the range of about 0.05 to about 5 MN/m². A tension within the former mentioned range generates a deformation Δl/l of the surface, preferably skin surface, to which it is applied, in the range of 0.1 to 0.4. This deformation range shows three phases, as illustrated in FIG. 13 (Diag.1). In phase A the skin extension under low tension is rapid, i.e. the skin shows a high elasticity of 0.1 to 0.3. In phase B the skin stiffens and is less elastic, being stretched by 0.3 to 0.35, followed by phase C, in which the skin is stiff and does not significantly deform even under higher tension (Δl/l from 0.35 to 0.4).

A skin deformation of higher than 0.4 is painful and hardly applicable, depending on the part of the body which it covers. As mentioned before, an effective skin stretching for the purpose of the present invention is preferably resulting in a deformation in the ranges A and B. The practitioner is also expected to perform the stretching of the skin along the direction in which is more stretchable than the other.

By stretching and deforming the surface of the skin, the concentration of pigments on the surface of the skin and the blood content in the vessels beneath the surface of the skin are reduced, resulting in the skin getting lighter in colour and absorbing less of the incident light radiation, hence avoiding a significant skin temperature increase.

The skin treatment is accordingly directed to the destruction of unwanted hairs (hair removal), coagulation of blood vessels of e.g. spider veins in the legs, treatment of pigmented skin either in the form of an erasure of dark sun stains or a tattoo removal. The hair removal with electromagnetic radiation is based on the selective energy absorption of the hair, which is darker than the surrounding skin. Accordingly, the pigments present in dark sun stains or tattoos selectively absorb the energy of the light beam.

The term “light beam” and “light” used throughout the specification interchangeably with the term “electromagnetic radiation” is not restricted to visible light of 400 nm to 700 nm. It merely defines wavelengths of the electromagnetic radiation ranging from 200 to 10600 nm, and the pulse duration of the light ranges from 1 nanosecond to 1 second, whereas the energy density of the light ranges is up to about 500 J/cm².

The radiation supplying means used according to the invention is preferably a laser or a source of intense pulsed light.

The light source is selected from the group of alexandrite laser, Nd:YAG laser, dye laser, erbium laser, CO₂ laser, diode laser, light emitting diode, excimer laser, ruby laser, Nd:YAG double frequency laser, Nd:glass laser, a non-coherent intense pulse light source, the latter also combined with an RF source.

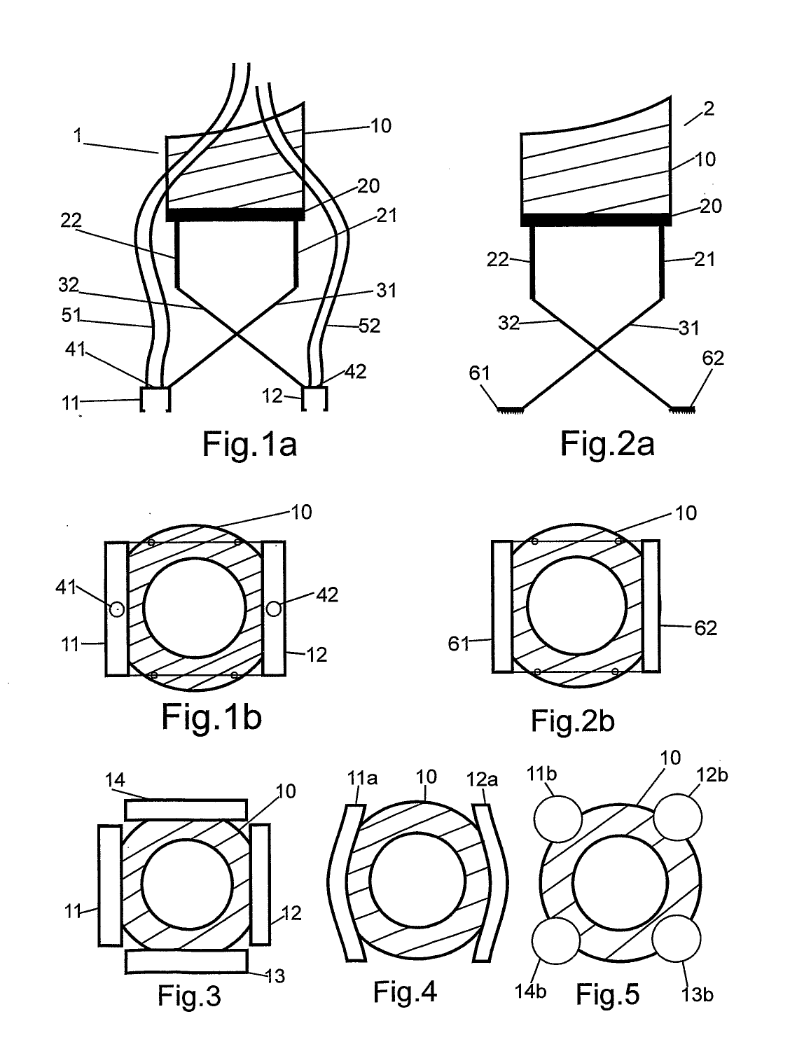

With reference to the accompanying drawings, FIG. 1a is a lateral view of the apparatus (1) according to one embodiment of the invention. The end portion (10) of the electromagnetic radiation supplying means (light source), is attached to a handpiece (20) comprising at its tip the anchoring means (21, 22, 31, 32, 11 and 12). The base of the handpiece (20) is generelly made of metal. It is ring-shaped or has any other form depending on the shape of the end portion (10) to which it is adapted. The anchoring means as shown in FIGS. 1a and 1b comprises relatively rigid metal rods (21 and 22) having a diameter of approximately 1 mm to 2 mm and a length of approximately 1 cm to 2 cm which continue as springs in the form of further, relatively thinner and flexible rods (31 and 32) positioned at an angle with respect to the rigid rods (21 and 22) to which they are welded.

Instead of the rigid metal rods (21, 22), metal tubes with an inner diameter of from 0.5 mm to 1 mm and an outer diameter of 1.2 mm to 2 mm can be used. The tubes are advantageous since they have an improved rigidity at the same external dimensions.

The flexible springs (31 and 32) are also made of metal, have a diameter of approximately 0.3 mm to 1.2 mm and a length of approximately 2 cm to 5 cm. The ring-shaped base of the handpiece (20), the rigid rods (21 and 22) and the flexible rods (31 and 32) are preferably made of stainless steel.

The angle between the rigid metal rods (21 and 22) and the flexible springs (rods 31 and 32) is obtuse and in the range of 110 to 160 degrees and preferably from 130 to 140 degrees.

The members (11 and 12) provided at the distal end of the flexible springs (31 and 32) and being positioned such as to face each other, are in the form of chambers (11 and 12) open at the bottom (in the direction of the targeted surface) and are connected to a vacuum pump via flexible tubes (51 and 52) at the respective openings (41 and 42).

The vacuum chambers (11, 12) are preferably made of synthetic material like polycarbonate, Plexiglas or polymethylmethacrylate (PMMA), since synthetic materials have the advantage of being bad thermal conductors. As will be explained below, according to preferred embodiments of the present invention the apparatus further comprises a cooling device in the form of a nozzle for supplying cooling means, e.g. air or a cooling composition, in the direction of the surface targetted by the light beam. Vacuum chambers made of a thermally conductive material like a metal would get too cold during operation of the apparatus and would, in contact with the skin moisture, freeze and get stuck on its surface, causing problems like painful frostbites. The dimensions of the vacuum chamber (11, 12) largely depend on the parts of the human body to which their application is intended and on the diameter of the light beam. Preferred dimensions are a length of 1 cm to 5 cm, a width of 0.2 cm to 1 cm and a height of 0.3 cm to 1.2 cm.

FIG. 1b is a bottom view of the above described embodiment.

The vacuum pump, which is not shown in these figures, generates sufficient negative pressure to anchor the vacuum chambers to the targeted surface and to stretch it for the duration of the irradiation. The vacuum is preferably automatically cut off between the irradiation pulses. For this reason, the vacuum is regulated by a valve driven by an optoelectronic controlling circuit which provides a synchronization with the light source and is switched on and cut off according to a signal provided by the optoelectronic controlling circuit.

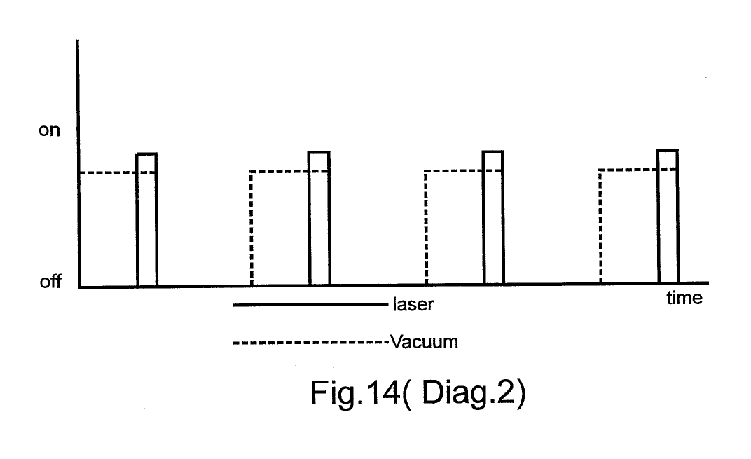

As soon as the light is interrupted, the interruption is monitored by a photodetector which communicates a signal to a controlling unit, according to which the vacuum is cut off. The time interval in which no vacuum is applied is shorter than the interval of interruption of the light. The vacuum is switched on well before the next light pulse starts. The synchronization of the intermittently applied vacuum with the cycles of light pulses emitted by the electromagnetic radiation supplying means (the light source) is diagrammatically shown in FIG. 14 (Diag.2).

By way of example, if the cycle of the light pulses is 750 ms and the time counted starts at the moment when the light is interrupted and the vacuum is cut off, the vacuum is switched on again after an interval of 500 ms and the light pulse starts 230 ms later. The light pulse has (for example) a duration of 20 ms and is then interrupted, simultaneously cutting off the vacuum. The cycle can be summarized as follows:

At time 0 (zero) the light is interrupted and the vacuum is cut off;

at 500 ms the vacuum is switched on;

at 730 ms light pulse starts;

at 750 ms the light pulse ends and the vacuum pump is switched off.

The cycle exemplified above enables the operator to remove the anchoring means from the already irradiated part and to reposition it on the skin surface before the next light pulse starts. Further, the pump is given a period of 230 ms to generate a sufficient vacuum and the operator stretches the targeted skin before the next light pulse starts.

The level of applied vacuum within the vacuum chambers need not be specified, since it is not constant; it gradually increases in the time interval during irradiation and is preferably automatically cut off after irradiation, in order to facilitate removal of the vacuum chambers from the skin surface.

In any case the level of applied vacuum during exposure to radiation is sufficient to anchor the periphery of the skin target and to stretch it in the direction of at least one of the two surface axes of the skin.

FIG. 2a is a lateral view of the apparatus (2) according to another embodiment of the invention, wherein the anchoring means of the handpiece (20) comprises at the distal end of the flexible springs (31 and 32) and instead of the vacuum chambers (11 and 12), anchoring members (61 and 62) comprising friction enhancing microprotusions, as e.g. in abrasive paper. In this embodiment the anchoring members (61 and 62) are also positioned such as to face each other.

FIG. 2b is a bottom view of the embodiment shown in FIG. 2a.

FIGS. 3, 4 and 5 are bottom views of other embodiments of the invention.

In FIG. 3 the plural anchoring means (11, 12, 13, 14) of the handpiece (20) are arranged as two couples, each comprising a first and a second member being positioned such as to face each other (11 and 12; 13 and 14).

In FIG. 4 the plural anchoring means comprises a first and a second member in curvilinear form (11a, 12a), being positioned such as to face each other.

In FIG. 5 the plural anchoring means (11b, 12b, 13b, 14b) are arranged as two couples, each comprising a first and a second member in circular form, being positioned such as to face each other.

The arrangements of the plural anchoring means shown in FIGS. 3, 4 and 5 can be related with either of the embodiments shown in FIGS. 1a, 1b, 2a and 2b, i.e. the members may be either in the form of vacuum chambers (11, 12) or as anchoring members comprising friction enhancing microprotusions (61, 62).

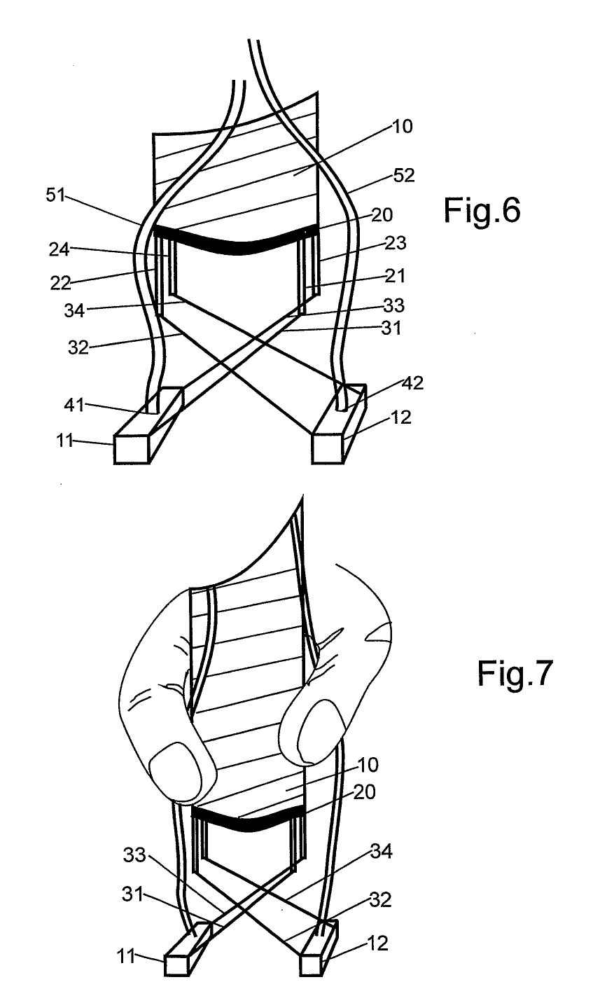

FIG. 6 is a three dimensional view of the apparatus according to the embodiment shown in FIGS. 1a and 1b. The handpiece (20) at the end portion (10) of the light source comprises at its tip the above mentioned anchoring means including the rigid metal rods (21, 22, 23, 24), the flexible springs (rods 31, 32, 33, 34) positioned at an angle with respect to the rigid rods, and the vacuum chambers (11, 12) open at the bottom (in the direction of the targeted surface) as connected to a vacuum pump via the flexible tubes (51 and 52) at the respective openings (41 and 42).

FIG. 7 is a three dimensional view of the same apparatus, as it is positioned by finger pressure on the skin target.

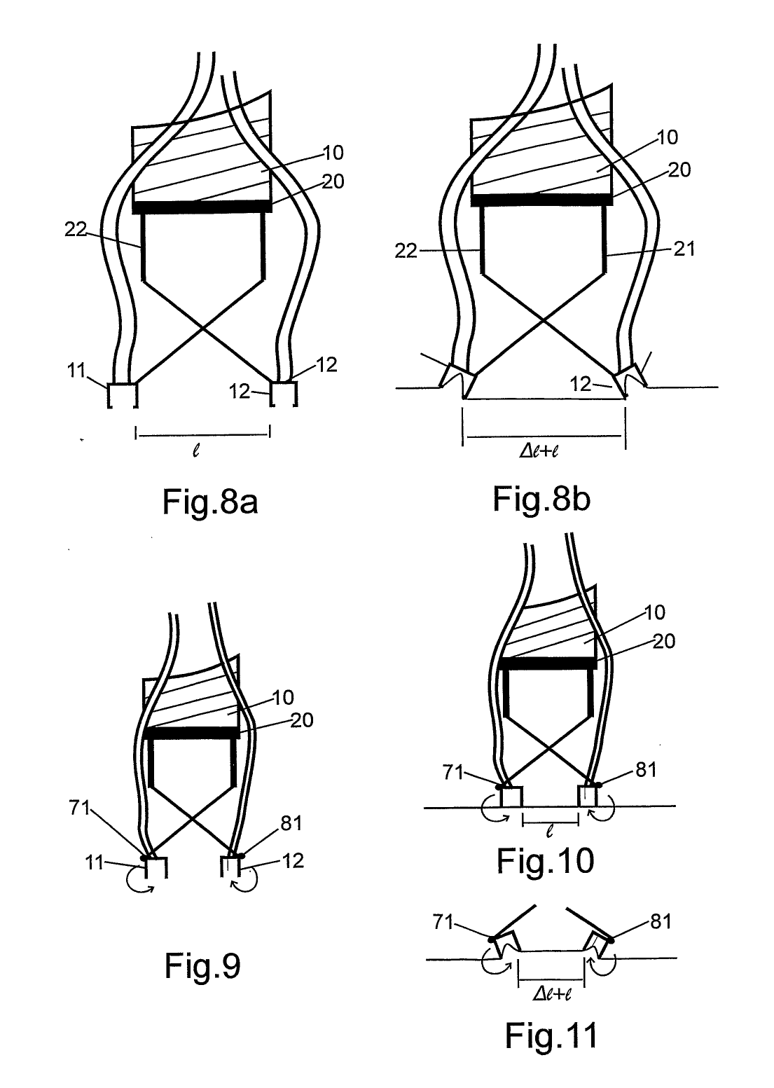

FIGS. 8a and 8b are simplified depictions of the apparatus as applied onto the surface targeted by the light source. FIG. 8a shows the handpiece immediately after its application to the skin and the initiation of the vacuum application. The skin is shown to be drawn and partly enter the vacuum chambers (11, 12). The targetted skin between the vacuum chambers is shown prior to its stretching and has a width dimension of l.

FIG. 8b shows the above embodiment after the skin target is stretched to a width of l+Δl during the application of vertical pressure and while the skin target is irradiated with electromagnetic radiation (light). It is seen that the pressure moves the anchoring means (i.e. the vacuum chambers 11, 12) sideways, i.e. in the direction of at least one of the axes of the skin surface and away from the targeted, radiation receiving part of said skin surface.

FIG. 9 is a lateral view of another embodiment, wherein the anchoring means is rotatable around an axis. According to this embodiment, the vacuum chambers (11, 12) are not fixedly attached to the flexible springs (rods 31, 32) but are allowed to rotate around an axis (71, 81) parallel to the length dimension of the chambers and the skin surface and perpendicular to the direction of deformation of either the flexible springs (31, 32) and the skin surface.

FIGS. 10 and 11 show the application of the latter embodiment to the skin target, prior and after the skin is stretched.

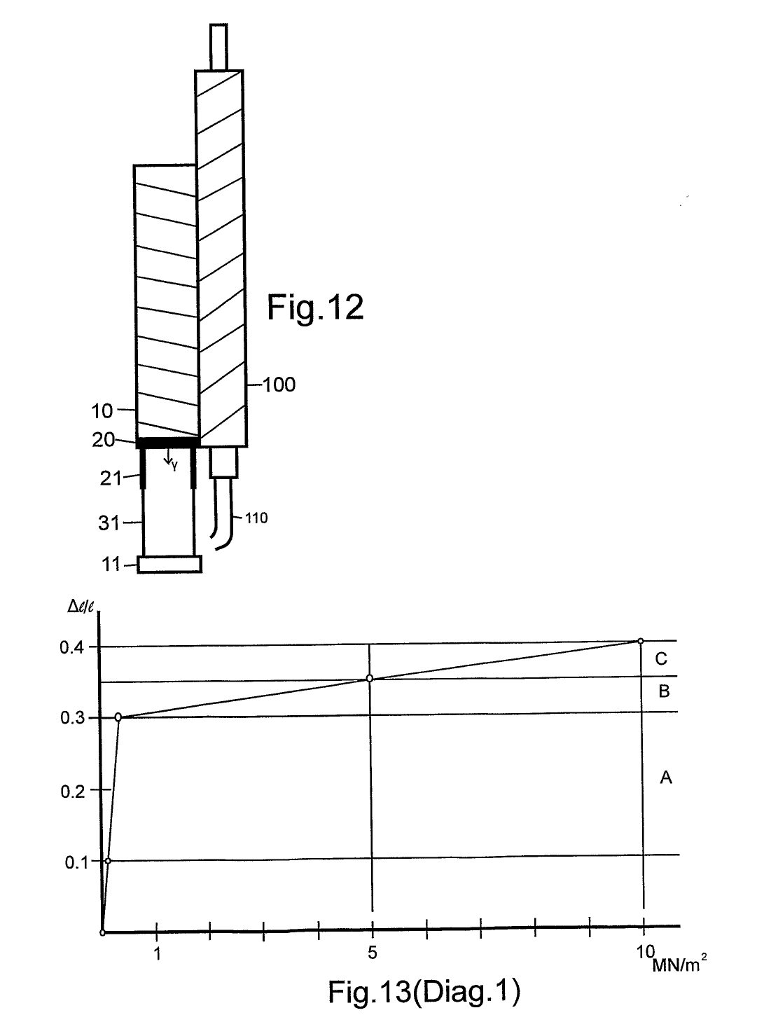

FIG. 12 is a lateral view of an embodiment of the apparatus according to the invention, wherein a major part of the apparatus is shown from the side normal to the depictions of FIGS. 1a, 2a, 8a, 8b, 9 and 10.

This figure shows the end portion (10) of the electromagnetic radiation source (light source) and the handpiece (20) comprising the rigid metal rods (only rod 21 is numbered), the flexible springs (only rod 31 is numbered) and the long side of the vacuum chamber (11). The flexible tubes (shown in the other drawings as 51, 52) and the vacuum pump are not shown. This drawing includes the cooling device (100) comprising a nozzle (110) for supplying cooling air to the skin target prior, during and after the irradiation with light.

All the above described embodiments of the apparatus and the method of the present invention provide a significant advantage over the prior systems in that the radiation receiving part of the targeted skin is not in direct contact with any part of the apparatus, being at a distance of at least a few millimeters, preferably at least 3 mm from the part where the light beam exits the optical path of the electromagnetic radiation supplying means, thus leaving enough space for the supply of cooling air.

The cooling device, which is preferably used in combination with all the above mentioned embodiments, supplies through a nozzle (110) a cooling means in the direction of said radiation receiving part. The cooling means is, in view of the drawbacks of using hydrofluorocarbons (HFC) as mentioned in the introduction above with respect to environmental issues, preferably cooled air. The cooling potential of air is sufficient for cooling the surface of the skin, when the skin surface has been tensioned and stretched, thus reducing the concentration of pigments on the surface of the skin and of the blood content in the vessels beneath the surface of the skin. As mentioned above, the reduction of the pigment concentration and the blood content per given area has the effect of the skin getting lighter coloured and absorbing less heat, thus significantly reducing the cooling needs.

By way of example, in a typical application a laser beam can raise the surface temperature of the skin by 50 degrees. Without cooling, the surface temperature is therefore raised from the normal 30° C. to 80° C. on the irradiated parts. In the case of cooling without skin stretching the cooling means shall drop the temperature by the same 50 degrees which are provided by the beam as heat source. Cooling by a hydrofluorocarbon like freon must therefore reduce the temperature by 50 degrees, resulting at a starting temperature of minus 20° C. and a final temperature of 30° C. In this case the starting temperature is too low to be comfortable to the patient, and the cooling action is not only superficial but is also propagated beneath the skin surface, being harmful to the skin. Furthermore, the energy needed for the skin treatment is in the range of over 25 J/cm² and the irradiation with light proves inefficient.

On the other hand, the stretching of the skin surface to be irradiated according to the present invention has the particular advantage of requiring less energy per given area for the same application, typically 15 to 18 J/cm², resulting in a lower increase of the temperature than in the prior art and hence requiring less cooling by the cooling means. As a further advantage, the cooling by a stream of cooled air has a pain relieving effect, being sufficiently anaesthetic.

Furthermore, in absence of an internally cooled transparent medium like glass in direct contact with the skin at the targetted position (which has the disadvantage of the glass surfaces becoming opaque due to the condensation of air humidity and the deposition of skin residues, affecting transmission properties of the glass and resulting in a significant absorption of the light emitted light source, disrupting the continuity of the irradiation process and being particularly time consuming and inefficient), the present invention provides a simple apparatus and a corresponding method for various skin treatments based on irradiation with electromagnetic radiation of a broad range of wavelengths and achieving an efficient workflow with reduced light energy consumption and being operable at reduced cooling requirements.

The present invention is described above only by embodiments that do not have limited effects on the scope of the appended claims, which define the scope of protection sought.

1. Apparatus for stretching a biaxially or radially deformable, resilient, flat or curved surface such as human skin comprising:
an electromagnetic radiation supplying means having an end portion (10) through which said radiation is supplied to a radiation receiving part of said surface, and
a handpiece (20) comprising at its tip at least one anchoring means to be applied to said surface said anchoring means being positioned laterally to said end portion and being operable to move in the direction of at least one of the axes of said biaxially or radially deformable flat or curved surface and away from said radiation receiving part of said surface.

2. Apparatus according to claim 1, wherein said anchoring means is operable to generate a tension in a direction of said surface in the range of about 0.01 to about 10 MN/m² (meganewton per square meter) and preferably in the range of about 0.05 to about 5 MN/m² .

3. Apparatus according to claim 1, wherein the radiation supplying means is a light source selected from the group of alexandrite laser, Nd:YAG laser, dye laser, erbium laser, CO₂ laser, diode laser, light emitting diode, excimer laser, ruby laser, Nd:YAG double frequency laser, Nd:glass laser, a non-coherent intense pulse light source, the latter also combined with an RF source or wherein the wavelength of the electromagnetic radiation ranges from 200 to 10600 nm, the pulse duration of the light ranges from 1 nanosecond to 1 second and the energy density of the light is up to about 500 J/cm².

4. Apparatus according to claim 1, wherein the radiation supplying means is a laser.

5. Apparatus according to claim 1, wherein the radiation supplying means is a source of intense pulsed light.

6. Apparatus according to claim 1, further comprising a cooling device (100) comprising a nozzle (110), for supplying cooling means, e.g. air or a cooling gaseous or spray composition, in the direction of said radiation receiving part of said surface.

7. Apparatus according to claim 1, wherein each of the at least one anchoring means of the handpiece comprises:
a pair of relatively rigid metal rods or tubes (21, 22) fixedly attached to a ring-shaped part of the handpiece (20) which perimetrically contacts the end portion (10) of the electromagnetic radiation supplying means,
a pair of springs (31, 32) in the shape of flexible, bendable rods, each welded to the distal end of each rigid metal rod or tube (21, 22) at an obtuse angle of from 110 to 160 degrees, and—an anchoring member (11, 12, 61, 62) to be applied to said surface.

8. Apparatus according to claim 1, wherein the anchoring member (11, 12) is rotatable around an axis (71, 81).

9. Apparatus according to claim 1, wherein the anchoring means comprises a vacuum applying means.

10. Apparatus according to claim 9, wherein the vacuum applying means comprises members having at least one chamber (11, 12), open in the direction of the surface and connected to a vacuum pump.

11. Apparatus according to claim 10, wherein the vacuum is regulated by a valve driven by an optoelectronic controlling circuit which provides a synchronization with the light source and is switched on and cut off according to a signal provided by the optoelectronic controlling circuit.

12. Apparatus according to claim 1, wherein the anchoring member (61, 62) comprises friction enhancing microprotusions, as e.g. in abrasive paper.

13. Apparatus according to claim 1, wherein the handpiece comprises plural anchoring means.

14. Apparatus according to claim 13, wherein the plural anchoring means are arranged in at least one couple each comprising a first and a second member (11, 12, 13, 14) being positioned such as to face each other on the opposite sides of said end portion of the electromagnetic radiation supplying means through which said radiation is supplied.

15. Apparatus according to claim 14, wherein said members are in rectilinear form (11, 12) or curvilinear form (11a, 12a).

16. Apparatus according to claim 14, wherein said members are in circular form (11b, 12b, 13b, 14b).

17. Apparatus according to claim 1, wherein the biaxially or radially deformable, resilient, flat or curved surface is skin, e.g. human skin.

18. Method for stretching a biaxially or radially deformable, resilient, flat or curved surface such as human skin comprising the steps of:
providing an electromagnetic radiation supplying means having an end portion (10) through which said radiation is supplied to a radiation receiving part of said surface,
providing a handpiece (20) at said end portion of the electromagnetic radiation supplying means, said handpiece comprising at its tip at least one anchoring means,
applying said at least one anchoring means to said surface, said anchoring means being positioned laterally to said end portion and further comprising the step of operating said at least one anchoring means to move in the direction of at least one of the axes of said biaxially or radially deformable flat or curved surface and away from said radiation receiving part of said surface.

19. Method according to claim 18, wherein said anchoring means generates a tension in a direction of said surface in the range of about 0.01 to about 10 MN/m² (meganewton per square meter) and preferably in the range of about 0.05 to about 5 MN/m² .

20. Method according to claim 18 further comprising the step of irradiating said radiation receiving part of said surface.

21. Method according to claim 18, wherein the radiation supplying means is a light source selected from the group of alexandrite laser, Nd:YAG laser, dye laser, erbium laser, CO₂ laser, diode laser, light emitting diode, excimer laser, ruby laser, Nd:YAG double frequency laser, Nd:glass laser, a non-coherent intense pulse light source, the latter also combined with an RF source or wherein the wavelength of the electromagnetic radiation ranges from 200 to 10600 nm, the pulse duration of the light ranges from 1 nanosecond to 1 second, the energy density of the light is up to about 500 J/cm².

22. Method according to claim 18, wherein the radiation supplying means is a laser.

23. Method according to claim 18, wherein the radiation supplying means is a source of intense pulsed light.

24. Method according to claim 18, also comprising a cooling process effected at least during the step of irradiating said radiation receiving part of said surface, wherein the cooling is provided by a cooling device (100) comprising a nozzle (110) supplying cooling means, e.g. air or a cooling gaseous or spray composition; in the direction of said radiation receiving part of said surface.

25. Method according to claim 18, wherein each of the at least one anchoring means of the handpiece comprises:
a pair of relatively rigid metal rods or tubes (21, 22) fixedly attached to a ring shaped part of the handpiece (20) which perimetrically contacts the end portion (10) of the electromagnetic radiation supplying means,
a pair of springs (31, 32) in the shape of flexible, bendable rods, each welded to the distal end of each rigid metal rod or tube (21, 22) at an obtuse angle of from 110 to 160 degrees, and
an anchoring member (11, 12, 61, 62) to be applied to said surface.

26. Method according to claim 18, wherein the anchoring member (11, 12) is rotatable around an axis (71, 81).

27. Method according to claim 18, wherein the anchoring means comprises a vacuum applying means.

28. Method according to claim 27, wherein the at least one vacuum applying means comprises members having at least one chamber (11, 12) open in the direction of the surface and connected to a vacuum pump.

29. Method according to claim 28, wherein the vacuum pump is regulated by a valve driven by an optoelectronic controlling circuit which provides a synchronization with the light source and is switched on and cut off according to a signal provided by the optoelectronic controlling circuit.

30. Method according to claim 18, wherein the anchoring member (61, 62) comprises friction enhancing microprotusions, as e.g. in abrasive paper.

31. Method according to claim 18, wherein the handpiece comprises plural anchoring means.

32. Method according to claim 31, wherein the plural anchoring means are arranged in at least one couple each comprising a first and a second member (11, 12, 13, 14) being positioned such as to face each other on the opposite sides of said end portion of the electromagnetic radiation supplying means through which said radiation is supplied.

33. Method according to claim 32, wherein said members are in rectilinear form (11, 12) or curvilinear form (11a, 12a).

34. Method according to claim 32, wherein said members are in circular form (11b, 12b, 13b, 14b).

35. Method according to claim 18, wherein the biaxially or radially deformable flat or curved surface is skin, e.g. human skin.

36. Use of an apparatus according to claim 1 for treatment of the skin.

37. Use according to claim 36 for hair removal, collagen contraction, photorejuvenation, treatment of vascular lesions, treatment of sebacouse or sweat glands, treatment of warts, treatment of pigmented lesions, treatment of damaged collagen, treatment of acne, treatment of warts, treatment of sweat glands, and treatment of psoriasis, and the vascular lesions are selected from the group of port wine stains, telangectasia, rosacea, and spider veins.
